(19) United States
(12) Patent Application Publication
(10) Pub. No.: US 2007/0288000 A1
(43) Pub. Date: Dec. 13, 2007

(54) Method for Aiding Valve Annuloplasty

(75) Inventor: Raoul Bonan, Mirabel (CA)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/737,294

(22) Filed: Apr. 19, 2007

Related U.S. Application Data
(60) Provisional application No. 60/793,269, filed on Apr. 19, 2006.

(51) Int. Cl. A61B 17/58; A61B 5/107; A61B 5/00

(52) U.S. Cl. 606/46; 600/587; 600/435; 600/585; 606/1

(57) ABSTRACT

The present invention provides methods for indirect imaging of the internal shape and structure of a heart. The invention can be practiced by injecting fluoroscopic contrast medium into the left atrium and ventricle of a heart, and by placing a radiopaque wire marker in the coronary sinus to provide a known reference location while fluoroscopic contrast medium is injected into the chambers of a heart. The invention provides for simultaneously injecting fluoroscopic contrast medium into both ventricles to characterize the ventricular septum and other cardiac structure. When using the disclosed methods, alone or in a combination with other methods and devices, a clinician can take multiple images of a heart in dyastole and systole. These images can be taken from different angles or the same angle and they can be superimposed upon each other to provide the clinician with a good image of the heart structure and the valve annulus.

### CROSS-REFERENCE TO RELATED APPLICATION

The present application claims priority to U.S. Provisional Application 60/793,269 filed Apr. 19, 2006 and titled “Method for Aiding Valve Annuloplasty; of which the entire contents of each are incorporated herein by reference.

### TECHNICAL FIELD

This invention relates generally to treating valvular regurgitation and particularly to a system and method for imaging the interior structure of a heart to aid in the implantation of a heart valve annuloplasty device by a catheter or minimally invasive surgery based method.

### BACKGROUND OF THE INVENTION

Heart valves, such as the mitral and tricuspid valves, are sometimes damaged by diseases or by aging, which can cause problems with the proper function of the valve. The mitral and tricuspid valves consist of leaflets attached to a fibrous ring or annulus. In a healthy heart, the mitral valve leaflets overlap during contraction of the left ventricle, or systole, and prevent blood from flowing back into the left atrium. However, due to various cardiac diseases, the mitral valve annulus may become distended, causing the leaflets to remain partially open during ventricular contraction and thus allowing regurgitation of blood into the left atrium. This results in reduced ejection volume from the left ventricle, causing the left ventricle to compensate with a larger stroke volume. The increased workload eventually results in dilation and hypertrophy of the left ventricle, further enlarging and distorting the shape of the mitral valve. If left untreated, the condition may result in cardiac insufficiency, ventricular failure, and death.

A common repair procedure involves implanting an annuloplasty device, such as an annuloplasty ring, on the superior, or atrial, surface of the mitral valve annulus. The annuloplasty ring is aligned with the valve annulus and then fixedly attached to the valve annulus. The annuloplasty ring generally has a smaller diameter than the distended valve annulus, and when attached to the annulus, the annuloplasty ring draws the annulus into a smaller configuration, bringing the mitral valve leaflets closer together and providing improved valve closure during systole.

Catheter-based repair procedures for implanting an annuloplasty device on the valve annulus require indirect visualization of, at least, the heart valve and annuloplasty device during placement of the device at the valve annulus. As used herein, the phrase indirect visualization refers to viewing an image of organs, body tissues, devices, and/or other structures within a patient while using x-ray, fluoroscopy, MRI, ultra-sound, or other known imaging modalities. Indirect visualization of a heart valve annulus is challenging. Cardiac tissue is not visible when using fluoroscopy, making it very difficult to accurately align a catheter delivered annuloplasty device prior to its implantation. In many procedures, radiopaque contrast dye is used with x-ray imaging equipment to increase the visualization of the area of interest.

One procedure that can be used to visualize/image the structure of a heart under fluoroscopy is contrast ventriculography. Contrast ventriculography is a procedure that is routinely performed in clinical practice during cardiac catheterization. Catheters must be intravascularly inserted within the heart, for example, to measure cardiac volume and/or flow rate. Ventriculograms are X-ray images that graphically represent the inner or endocardial surface of the ventricular chamber. These images are typically used to determine tracings of the endocardial boundary at end diastole (ED), when the heart is filled with blood, and at end systole (ES), when the heart is at the end of a contraction during the cardiac cycle. By manually tracing the contour or boundary of the endocardial surface of the heart at these two extremes in the cardiac cycle, a physician can determine the size and function of the left ventricle and can diagnose certain abnormalities or defects in the heart. Of the end systole and end diastole images, the former is perhaps the most useful in diagnosing cardiac abnormalities.

To produce a ventriculogram a radiopaque contrast fluid is injected into the left ventricle (LV) of a patient's heart. An X-ray source is aligned with the heart, producing a projected image representing, in silhouette, the endocardial surface of the heart (myocardium) muscle. The silhouette image of the LV is visible because of the contrast between the radiopaque fluid and other surrounding physiological structure.

While ventriculography works well for determining things like the ejection fraction of the left ventricle, it has not proven to be practical for things such as visualizing/imaging the mitral valve annulus during catheter based implantation of a device for treating MR. When treating the mitral valve, the rapid wash-out in high-flow area being imaged requires multiple injections of the fluoroscopic contrast medium for a clinician using the previously known methods to verify the location of the annulus with sufficient certainty required. Thus, multiple injections of high volumes of fluoroscopic contrast medium would be required to gain sufficient knowledge of the mitral valve shape and orientation. These multiple high-volume contrast injections are not desirable for the patient due to potential complications in the renal system, where the radiopaque fluoroscopic contrast medium is filtered from the blood.

Catheter based annuloplasty procedures are further challenged by the structure of the valve annulus. In particular, the mitral valve annulus lacks a definable shelf or ledge for conveniently locating an annuloplasty device. The mitral valve leaflets are little more than flaps or appurtenances attached to the cardiac muscle tissue, creating a pseudo-annulus. During systole, the mitral valve is closed to form a relatively flat floor of the left atrium. However, during diastole, the mitral valve leaflets open towards the ventricular walls such that, in many cases, the valve annulus is not well defined. Since annuloplasty is performed on a beating heart, care must be taken during both systole and diastole when positioning an annuloplasty device for fixation.

Without the direct optical visualization that is provided during surgery, it is difficult to position an annuloplasty device in abutment with the superior surface of the valve annulus. With indirect imaging techniques used during a catheter-based procedure, an annuloplasty device may be inadvertently affixed in a misaligned position above or angled across the valve annulus. Affixing the annuloplasty device in such a misaligned position could have negative consequences for the patient, such as increasing mitral regurgitation and/or triggering ectopic heart beats.

Other techniques for viewing images of cardiac structures include ultrasonography such as trans-thoracic echocardiography (TTE), trans-esophageal echocardiography (TEE), and cardiac magnetic resonance (CMR) including magnetic resonance imaging (MRI) or magnetic resonance angiography (MRA). However, the level of detail concerning valve structure that is provided by the above techniques, used alone or in combination could be improved to allow a clinician to accurately locate the valve structure during catheter-based valve repair procedures.

Therefore, it would be desirable to provide a system and method for aiding implantation of an annuloplasty device to overcome the aforementioned and other disadvantages.

### SUMMARY OF THE INVENTION

The present invention provides methods for visualization of the heart during therapeutic procedures. An example of such procedures would be repair of cardiac valves. A specific example would be catheter-based or other minimally invasive annuloplasty device implantation to treat mitral regurgitation. Thus the current invention will be described herein in terms of use for visualization of the structure in a heart in preparation for or during a catheter based procedure to implant an annuloplasty device on the mitral valve annulus while the heart is still beating. Those skilled in the art will readily understand that the methods disclosed herein could be used for indirect visualization of the heart structure for other purposes as well.

One object of the current invention is to provide methods that can be used to assist in the indirect visualization of targeted areas in the cardiac structure, such as the mitral valve.

Another object of the current invention is to provide devices that can be used as reference devices during the indirect visualization of the cardiac structure such that the location of the mitral valve annulus can be identified or predicted. Examples of such structure would be the coronary sinus, the left circumflex artery, and other vessels in the coronary vasculature.

One aspect of the present invention provides for puncturing the septum between the right and left atria of a heart, advancing a catheter to the left atrium and injecting a fluoroscopic contrast medium into the left atrium. This will allow a clinician to visualize the shape and size of the left atrium, and assist in determining the location of the mitral valve and the mitral valve annulus.

Another aspect of the present invention provides for advancing a catheter to the left ventricle via the aorta, and injecting a fluoroscopic contrast medium into the ventricle just under the mitral valve leaflets. This will allow a clinician to visualize the shape, the size, and the plane of the mitral valve and the mitral valve annulus.

Yet another aspect of the invention provides for placing a radiopaque wire marker in the coronary sinus to provide a known reference location while fluoroscopic contrast medium is injected into the chambers of a heart.

A further aspect of the invention provides for advancing separate catheters into the left and right ventricles of a heart and simultaneously injecting a fluoroscopic contrast medium into the ventricles. This will allow a clinician to visualize the shape and structure of the ventricles and to identify the location and thickness of the septal wall that separates the ventricles.

When using the disclosed methods, alone or in a combination with other methods and devices, a clinician can take multiple images of a heart in dyastole and systole. These images can be taken from different angles or the same angle and they can be superimposed upon each other to provide the clinician with a good image of the heart structure and the location of a valve annulus.

The aforementioned and other features and advantages of the invention will become further apparent from the following detailed description of the presently preferred embodiments, read in conjunction with the accompanying drawings, which are not to scale. The detailed description and drawings are merely illustrative of the invention rather than limiting, the scope of the invention being defined by the appended claims and equivalents thereof.

### BRIEF DESCRIPTION OF THE DRAWINGS

FIG. 1 is an illustration showing a cross-sectional interior view of a human heart;

FIG. 2 is an illustration showing a cross-sectional interior view of a human heart with catheters and devices inserted into the chambers and structure of the heart;

FIG. 3 is another illustration showing a cross-sectional interior view of a human heart with catheters and devices inserted into the chambers and structure of the heart;

FIG. 4 is another illustration showing a cross-sectional interior view of a human heart with catheters and devices inserted into the chambers and structure of the heart;

FIG. 5 is a block diagram showing a method for visualization of the interior structure of a heart, according to the current invention; and

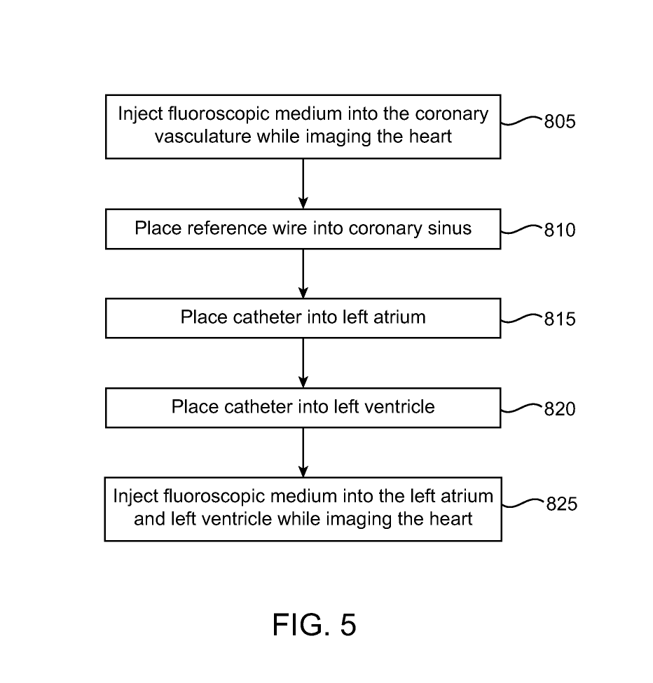

FIG. 6 is a block diagram showing a method for visualization of the interior structure of a heart, according to the current invention.

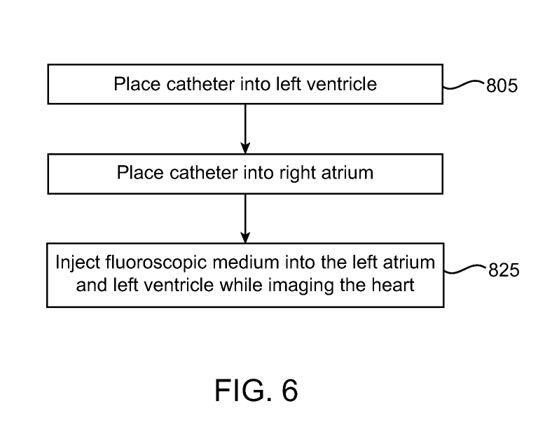

### DETAILED DESCRIPTION

The invention will now be described by reference to the figures wherein like numbers refer to like structures. The terms “distal” and “proximal” are used herein with reference to the treating clinician during the use of the catheter system; “Distal” indicates an apparatus portion distant from, or a direction away from the clinician and “proximal” indicates an apparatus portion near to, or a direction towards the clinician.

The catheters used for practicing the current invention are flexible, and configured so that they can be inserted into the cardiovascular system of a patient. Appropriate catheters are made of flexible biocompatible materials such as polyurethane, polyethylene, nylon and polytetrafluoroethylene (PTFE).

The catheters and devices of the current invention may be made, in whole or in part, from one or more materials that are viewable by radiography, ultrasound, or magnetic resonance imaging visualization techniques. Embodiments of the devices may also be coated with materials that are visible using such visualization methods.

Some embodiments of the devices that are viewable by radiography can include materials having a high X-ray attenuation coefficient (radiopaque materials). The devices may be made in whole or in part from the material, or they may be coated in whole or in part by radiopaque materials. Alloys or plastics may include radiopaque components that are integral to the materials. Examples of suitable radiopaque material include, but are not limited to gold, tungsten, silver, iridium, platinum, barium sulfate and bismuth sub-carbonate.

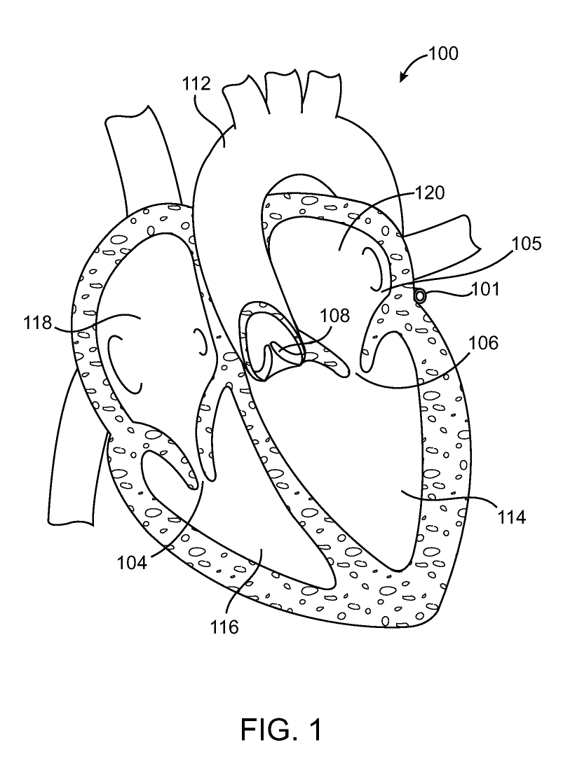

Referring to the drawings, FIG. 1 is an illustration of the interior of a human heart 100. The heart 100 includes four valves that work in synchrony to control the flow of blood through the heart. The tricuspid valve 104 situated between the right atrium 118 and the right ventricle 116; and the mitral valve 106, located between the left atrium 120 and the left ventricle 114, facilitate filling of ventricles 116 and 114 on the right and left sides, respectively, of the heart 100. Also seen in the figure is the coronary sinus 101 that is typically located in the atrio-ventricular groove, and generally on the same plane as the mitral valve annulus.

The aortic valve 108 is situated at the junction between the aorta 112 and the left ventricle 114 and facilitates blood flow from heart 100, through the aorta 112 to the peripheral circulation. The Pulmonary valve is situated at the junction of right ventricle 116 and pulmonary artery. The pulmonary valve facilitates blood flow from heart 100 through the pulmonary artery to where the pulmonary artery forms left and right branches that carry blood to the left and right lungs respectively.

The four valves work by opening and closing in harmony with each other. During diastole, the tricuspid valve 104 and mitral valve 106 open and allow blood flow into the ventricles 114 and 116, and the pulmonic valve and aortic valve are closed. During systole, shown in FIG. 1, the aortic valve 108 and pulmonary valve open and allow blood flow from left ventricle 114, and right ventricle 116 into the aorta 112 and pulmonary artery, respectively.

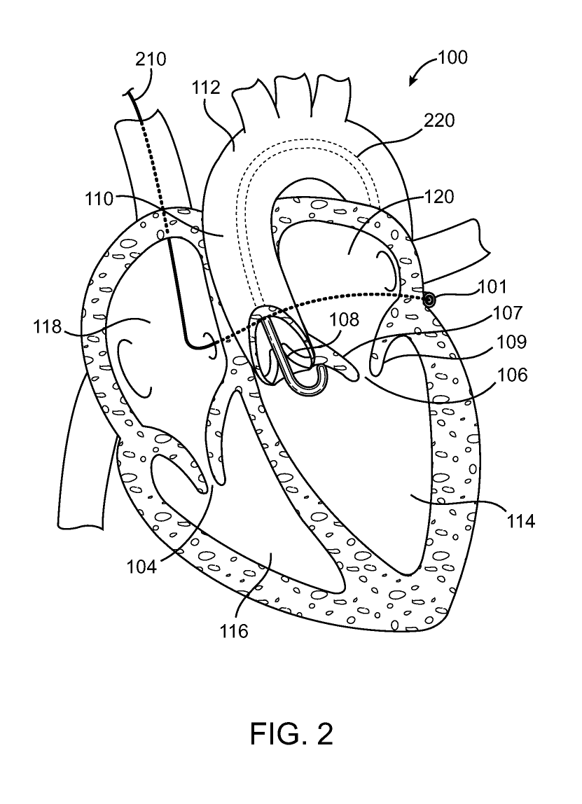

FIGS. 2-4, are illustrations showing hearts with catheters and wires inserted for the indirect imaging methods for identifying the size, shape, and orientation of cardiac structure according to the teachings of the current invention. The current invention uses fluoroscopy, which is familiar to clinicians for use as an imaging method in the diagnosis and treatment of coronary artery disease. Placing reference structures at key locations within the heart can provide a visual aid to allow precise navigation through the open chambers of the heart. The annulus of the mitral valve can be characterized in terms of size, location and plane using the techniques disclosed herein. The valvular papillary muscles, leaflets and septum can be characterized similarly.

Referring to FIG. 2, one method for imaging the mitral valve provides for advancing a radiopaque wire 210 into the coronary vasculature and preferably into the coronary sinus 101. Navigation through the vasculature to the coronary sinus can be done via routes known to those having ordinary skill in the art. An angiographic catheter 220 having a curved distal end is advanced through the aorta 112 to the left ventricle 114 and positioned such that the tip of the catheter is adjacent to the wall of the ventricle and just below the mitral valve annulus 105. The catheter can be inserted into and advanced through the femoral artery into the aorta, through the aortic valve into the left ventricle.

The distal portion of the catheter is curved, and in one embodiment of the invention a catheter having a standard Judkins curve is used. Once the catheter is positioned, a fluoroscopic contrast medium is injected under the valve annulus. The fluoroscopic contrast medium remains under the valve annulus, and some of the agent remains under the annulus for sufficient time to allow a clinician to locate the plane and general shape of the annulus before the fluoroscopic contrast medium is flushed from the heart.

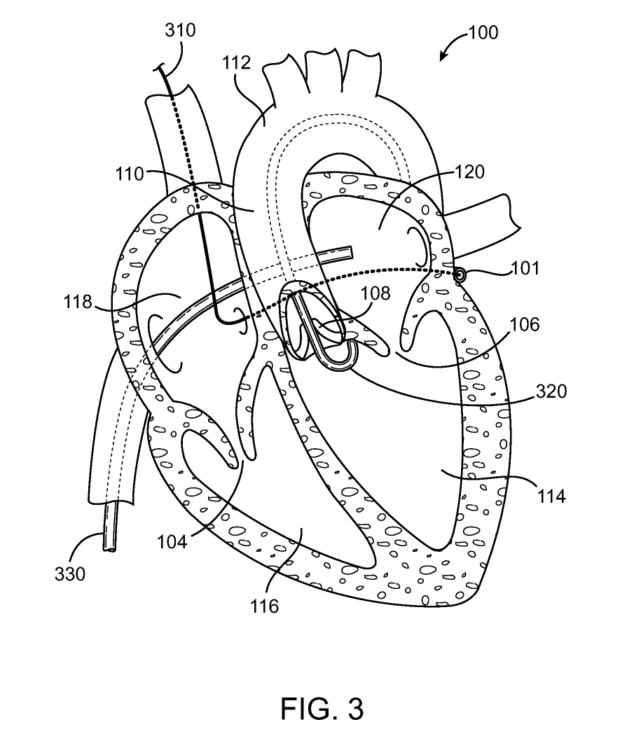

FIG. 3 illustrates another method for imaging of the mitral valve 106 structure in a heart 100 according to the current invention. To practice the method of the FIG. a clinician would advance a catheter into the coronary vasculature and inject a fluoroscopic contrast medium to identify the location of key vessels, such as the coronary sinus 101, in the vasculature relative to the A-V groove. A radiopaque reference wire 310 is then advanced to the coronary sinus. A catheter 330 having a straight distal portion is advanced to the right atrium 118, through the septum, and into the left atrium 120. An angiographic catheter 320 having a curved distal end is advanced into the left ventricle 114 via the aorta 112. The angiographic catheter 120 in the ventricle is then manipulated so that the tip of the catheter is directly under, and in close proximity to the mitral valve annulus. Fluoroscopic contrast medium is then injected into the left atrium 120 and under the mitral valve annulus in the left ventricle 114 while the heart is being imaged. Having contrast medium in the left atrium and under the mitral valve annulus in the left ventricle will allow a clinician to determine the location of the mitral valve annulus.

The fluoroscopic contrast medium injected under the mitral valve annulus, will remain under the annulus long enough to allow a clinician to obtain an accurate image of the location and orientation of the annulus. Based on the angle from which an image is taken, the clinician may also be able to obtain knowledge regarding the shape and size of the annulus.

FIG. 3 illustrates delivery of the catheter 330 into the left atrium via a transeptal approach through the vena cava. To take this approach, the catheter 330 is inserted through the femoral vein into the common iliac vein, through the inferior vena cava into the right atrium 118. The transeptal wall 142 between the right atrium 118 and left atrium 120 is then punctured (preferably at the fossa ovalis) with a guide wire or other puncturing device, and the distal end of the catheter advanced into the left atrium 120.

Those skilled in the art will appreciate that alternative paths to gain access to the left atrium are available. For example, another possible path would be through the radial vein into the brachial vein, through the subclavian vein, through the superior vena cava into the right atrium, and then transeptally into the left atrium. Yet another possible path would be through the femoral artery into the aorta, through the aortic valve into the left ventricle, and then retrograde through the mitral valve into the left atrium.

Several standard views were identified to aid in the spatial mapping of the chambers and structures of the heart. For example, in the porcine model, a left ventriculogram at 10CRA-70RAO provides a good view of the papillary muscles and a good view of the mitral valve. In humans, views perpendicular to the Mitral Annulus LAO 45 Caudal 25 and parallel to the Mitral Annulus RAO 45 Cranial 20 would allow a clinician to adequately determine the interior structure of the heart. However, the views in humans may need to be tailored based on a coronary angiogram to determine the location of the left coronary descending artery, the right coronary artery, and the coronary sinus relative to the A-V groove.

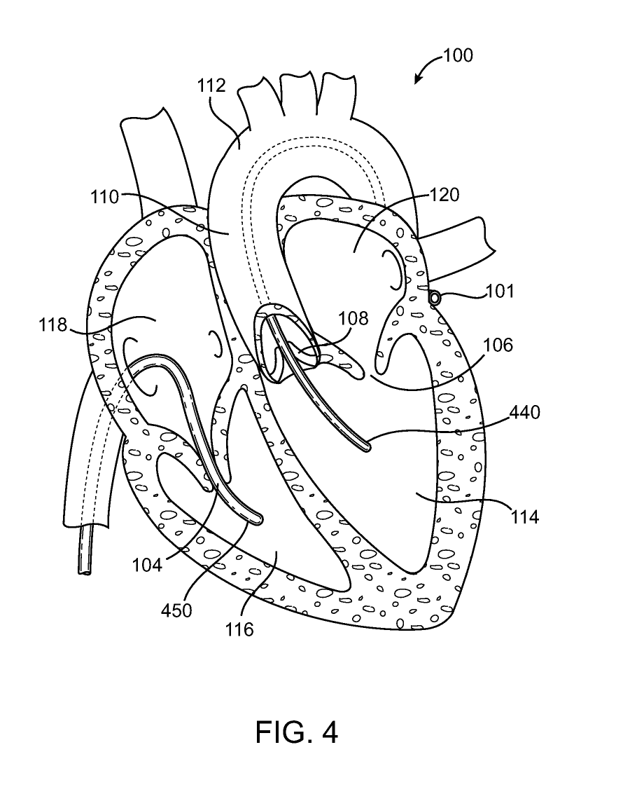

FIG. 4 illustrates another aspect of the current invention, which is method for determining the location and thickness of the septum between the left and right ventricles of a heart. FIG. 4 shows a first catheter 440 having a straight distal portion in the left ventricle 114 of heart 100 and a second catheter 450 having a straight distal portion in the right ventricle 116. The first catheter is advanced through the aorta 112 as described above and the catheter in the right ventricle can be advanced to the right atrium 118 as described above, and then through the tricuspid valve 104 and into the ventricle.

Fluoroscopic contrast medium is then simultaneously injected into both ventricles while the heart is being imaged. Based on the angle from which an image is taken, a clinician may also be able to obtain knowledge regarding the thickness of the septum, the location of the papillary muscles, and other information about the structure of the heart.

Using the methods disclosed herein allows a clinician to identify key reference structures of the heart using currently available technology in a novel and non-obvious way. For example, the annulus of a mitral valve can be characterized in terms of size, location and plane using the disclosed methods. The valvular papillary muscles, leaflets and septum can be similarly characterized and these parts of the cardiac structure can be mapped/characterized relative to the other. Additionally, mapping can be conducted using devices such as stents, guidewires, and angiography catheters. These methods used alone or in combination with other methods, support the development and use of new minimally invasive, and catheter based, treatments desired by clinicians and patients alike. Once the shape, size orientation and/or other characteristics of the heart structure have been determined, a clinician can then use the information gained from the methods disclosed herein to implant a therapeutic device, such as a catheter delivered annuloplasty device, in the heart.

When using the disclosed methods, alone or in a combination with other methods and devices, a clinician can take multiple images of a heart in dyastole and systole. These images can be taken from different angles or the same angle and they can be superimposed upon each other to provide the clinician with a good image of the heart structure and the location of a valve annulus. Once the shape, size orientation and/or other characteristics of the heart structure have been determined, a clinician can then use the information gained from the methods disclosed herein to implant a therapeutic device, such as a catheter delivered annuloplasty device, in the heart.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A method for imaging heart structure before and during procedures for providing therapeutic treatment, the method comprising the steps of:
delivering an elongated reference wire to the coronary sinus;
delivering at least one elongated tubular catheter having a distal end and a proximal end to a desired chamber within the heart;
positioning the at least one catheter in a desired location within the desired heart chamber; and
injecting a fluoroscopic contrast medium from the at least one catheter while viewing the heart from at least one angle.

2. The method of claim 1 wherein at least a portion the elongated reference wire is made from a radiopaque material.

3. The method of claim 1 wherein at least a portion of the elongated reference wire is coated with a radiopaque material.

4. The method of claim 1 wherein the step of delivering at least one catheter to a desired chamber within the heart comprises delivering an angiographic catheter having a curved distal portion to the left ventricle.

5. The method of claim 4 wherein the step of positioning the at least one catheter in a desired location within the desired heart chamber comprises positioning the distal portion of the angiographic catheter against the wall of the left ventricle such that a distal tip of the catheter is directly under the mitral valve annulus.

6. The method of claim 1 wherein the step of delivering at least one catheter to a desired chamber within the heart comprises delivering a first angiographic catheter having a curved distal portion to the left ventricle and delivering a second catheter having a straight distal portion to the left atrium.

7. The method of claim 6 wherein the second catheter is delivered to the left atrium by routing the catheter into the right atrium and through a puncture location in the fossa ovalis.

8. The method of claim 6 wherein the step of positioning the at least one catheter in a desired location within the desired heart chamber comprises positioning the distal portion of the first angiographic catheter against the wall of the left ventricle such that the distal tip of the catheter is directly under the mitral valve annulus and positioning a distal tip of the second catheter inside the left atrium.

9. A method for imaging heart structure before and during procedures for providing therapeutic treatment, the method comprising the steps of:
delivering an elongated reference wire to the coronary sinus;
delivering a first elongated tubular catheter having a proximal end, and a curved distal portion to the left ventricle of the heart;
delivering a second elongated tubular catheter having a proximal end and a straight distal portion to the left atrium;
positioning a distal tip of the first catheter at a desired location within the left ventricle;
positioning a distal tip of the second catheter within the right atrium chamber; and
injecting a fluoroscopic contrast medium from the first and second catheters at the same time while viewing the heart from at least one angle.

10. The method of claim 9 wherein at least a portion the elongated reference wire is made from a radiopaque material.

11. The method of claim 9 wherein at least a portion of the elongated reference wire is made for a material selected from the group consisting of: gold, tungsten, silver, iridium, platinum, barium sulfate and bismuth sub-carbonate.

12. The method of claim 9 wherein at least a portion of at least one of the first catheter or the second catheter is coated with a radiopaque material selected from the group consisting of: gold, tungsten, silver, iridium, platinum, barium sulfate and bismuth sub-carbonate.

13. The method of claim 9 wherein the step of positioning a distal tip of the first catheter at a desired location within the left ventricle comprises positioning the distal portion of the angiographic catheter against the wall of the left ventricle such that the distal tip of the catheter is directly under the mitral valve annulus.

14. The method of claim 9 wherein while fluoroscopic contrast medium is being injected from the catheters, the procedure is viewed from an angle above the mitral valve annulus.

15. The method of claim 9 wherein while fluoroscopic contrast medium is being injected from the catheters, the procedure is viewed from an angle parallel to the mitral valve annulus.

16. The method of claim 9 wherein the first catheter is delivered to the left ventricle by routing the catheter through the aorta and past the aortic valve, and the second catheter is delivered to the left atrium by routing the catheter into the right atrium and through a puncture location in the fossa ovalis.

17. The method of claim 9 comprising the additional step of repeating the injection of fluoroscopic contrast medium from the first and second catheters while recording multiple fluoroscopic images of the heart from more than one angle.

18. A method for imaging heart structure before and during procedures for providing therapeutic treatment, the method comprising the steps of:
delivering a first elongated tubular catheter having a proximal end, and a straight distal portion to the left ventricle of the heart;
delivering a second elongated tubular catheter having a proximal end and a straight distal portion to the right ventricle of the heart;
positioning a distal tip of the first catheter within the left ventricle;
positioning a distal tip of the second catheter within the right ventricle; and
simultaneously injecting a fluoroscopic contrast medium from the first and second catheters while viewing the heart from at least one angle.

19. The method of claim 18 comprising the additional step of delivering an elongated reference wire to the coronary sinus wherein at least a portion of the reference wire is made from a radiopaque material.

20. The method of claim 18, wherein at least a portion of at least one of the first catheter and at least a portion of the second catheter is coated with a radiopaque material selected from the group consisting of: gold, tungsten, silver, iridium, platinum, barium sulfate and bismuth sub-carbonate.
